(19) United States
(12) Patent Application Publication
(10) Pub. No.: US 2022/0395511 A1
(43) Pub. Date: Dec. 15, 2022

(54) COMPOSITIONS AND METHODS FOR INCREASING CELL SURFACE OXYTOCIN RECEPTOR (OXTR)

(71) Applicant: Washington University, St. Louis, MO (US)

(72) Inventors: Sarah England, St. Louis, MO (US); Manasi Malik, St. Louis, MO (US); Princess Imoukhuede, St. Louis, MO (US); Yingye Fang, St. Louis, MO (US)

(73) Assignee: Washington University, St. Louis, MO (US)

(21) Appl. No.: 17/837,224

(22) Filed: Jun. 10, 2022

Related U.S. Application Data
(60) Provisional application No. 63/209,054, filed on Jun. 10, 2021.

(51) Int. Cl. A61K 31/5355; A61K 31/166; A61K 31/4709; A61K 31/55

(52) U.S. Cl. CPC A61K 31/5355; A61K 31/166; A61K 31/4709; A61K 31/55

(57) ABSTRACT

Among the various aspects of the present disclosure is the provision of OXTR chaperones and methods of use thereof. An aspect of the present disclosure provides for a method of increasing the display of oxytocin receptor (OXTR) on a plasma membrane in a cell of a subject comprising: administering an OXTR chaperone, wherein the OXTR chaperone increases OXTR on a cell surface. Another aspect of the present disclosure provides for a method of increasing or restoring oxytocin sensitivity in a subject comprising: administering an OXTR chaperone, wherein the OXTR chaperone increases OXTR on a cell surface. Yet another aspect of the present disclosure provides for a method of increasing the efficacy of oxytocin or synthetic oxytocin in a subject comprising: administering an OXTR chaperone, wherein the OXTR chaperone increases OXTR on a cell surface.

### CROSS-REFERENCE TO RELATED APPLICATIONS

This reference claims the benefit of U.S. Provisional Patent Application No. 63/209,054, filed Jun. 10, 2021, the entire disclosure of which is incorporated herein by reference.

### STATEMENT REGARDING FEDERALLY SPONSORED RESEARCH OR DEVELOPMENT

This invention was made with government support under HD096737 and HD097525 awarded by the National Institutes of Health. The government has certain rights in the invention.

### FIELD

The present disclosure generally relates to increasing surface cell oxytocin receptors (OXTRs).

### BACKGROUND OF THE INVENTION

The nonapeptide hormone oxytocin modulates social behavior, mediates the lactation reflex, and induces and strengthens uterine contractions. Synthetic oxytocin is administered to induce or augment labor and prevent postpartum hemorrhage in a large portion of patients who give birth. However, response to oxytocin varies widely between individuals. Inadequate response to oxytocin poses significant clinical challenges, including labor arrest, requirement for Cesarean section, and postpartum hemorrhage, which can increase risk for complications and lead to maternal mortality. Thus, it would be clinically advantageous to develop strategies to improve oxytocin response in laboring patients.

One strategy could be to identify pharmacological chaperones (pharmacoperones) that increase cell surface localization and function of the oxytocin receptor (OXTR), which is a G-protein-coupled receptor (GPCR). Studies on other GPCRs show that antagonists can increase cell surface levels of WT or variant receptors. For instance, long-term treatment with p adrenergic receptor antagonists (3 blockers) increases receptors on the cell surface, leading to excessive p adrenergic stimulation when p blockers are withdrawn. Antagonist pharmacoperones appear to act by promoting anterograde transport of receptors to the cell surface: they permeate the cell membrane and bind to immature receptors either during or after translation, thus stabilizing the native state of the protein).

Pharmacoperones have also been investigated for therapeutic use in patients with genetic variants in the arginine vasopressin receptor 2 (AVPR2), a GPCR with high similarity to OXTR. Pathogenic variants impair AVPR2 folding and trafficking to the cell surface, resulting in nephrogenic diabetes insipidus. Previous studies have shown that antagonists, agonists, and allosteric ligands are effective in rescuing AVPR2 trafficking and function: one such antagonist, SR49059 ((2S)-1-[[(2R,3S)-5-Chloro-3-(2-chlorophenyl)-1-[(3,4-dimethoxyphenyl)sulfonyl]-2,3-dihydro-3-hydroxy-1H-indol-2-yl]carbonyl]-2-pyrrolidinecarboxamide), showed promise in a small clinical trial of patients with nephrogenic diabetes insipidus. However, no compounds that act as OXTR pharmacoperones have previously been described.

### SUMMARY

Among the various aspects of the present disclosure is the provision of OXTR chaperones and methods of use thereof. An aspect of the present disclosure provides for a method of increasing the display of oxytocin receptor (OXTR) on a plasma membrane in a cell of a subject comprising: administering an OXTR chaperone, wherein the OXTR chaperone increases OXTR on a cell surface. Another aspect of the present disclosure provides for a method of increasing or restoring oxytocin sensitivity in a subject comprising: administering an OXTR chaperone, wherein the OXTR chaperone increases OXTR on a cell surface. Yet another aspect of the present disclosure provides for a method of increasing the efficacy of oxytocin or synthetic oxytocin in a subject comprising: administering an OXTR chaperone, wherein the OXTR chaperone increases OXTR on a cell surface. In some embodiments, the method further comprises administering oxytocin or a derivative thereof, such as synthetic oxytocin (e.g., Pitocin) to the subject. In some embodiments, the OXTR chaperone is selected from an OXTR binding agent (e.g., OXTR antagonist) or vasopressin inhibitor. In some embodiments, the OXTR chaperone is an OXTR antagonist, L371,257 (1-[1-[4-(1-acetyl piperidin-4-yl)oxy-2-methoxybenzoyl]piperidin-4-yl]-4H-3,1-benzoxazin-2-one). In some embodiments, the OXTR chaperone is a vasopressin inhibitor, SR49059 ((2S)-1-[[(2R,3S)-5-Chloro-3-(2-chlorophenyl)-1-[(3,4-dimethoxyphenyl)sulfonyl]-2,3-dihydro-3-hydroxy-1H-indol-2-yl]carbonyl]-2-pyrrolidinecarboxamide). In some embodiments, the OXTR chaperone is a vasopressin inhibitor, OPC 21268 (N-[3-[4-[[4-(3,4-dihydro-2-oxo-1(2H)-quinolinyl)-1-piperidinyl]carbonyl]phenoxy]propyl]-acetamide). In some embodiments, the OXTR chaperone is a vasopressin inhibitor, SSR 149415 ((2S,4R)-1-[(3R)-5-Chloro-1-[(2,4-dimethoxyphenyl)sulfonyl]-2,3-dihydro-3-(2-methoxyphenyl)-2-oxo-1H-indol-3-yl]-4-hydroxy-N,N-dimethyl-2-pyrrolidinecarboxamide). In some embodiments, the OXTR chaperone is a vasopressin inhibitor, tolvaptan (N-(4-{[(5R)-7-Chloro-5-hydroxy-2,3,4,5-tetrahydro-1H-1-benzazepin-1-yl]carbonyl}-3-methylphenyl)-2-methylbenzamide; also known as OPC41061). In some embodiments, increasing the display of OXTRs comprises increasing the trafficking of the receptors from intracellular stores (in the endoplasmic reticulum and Golgi body) to the cell surface. In some embodiments, the OXTR chaperone is administered in an amount effective to: improve trafficking of the oxytocin receptor; enhance clinical response to oxytocin; increase uterine contractions during childbirth; induce or augment labor; prevent or reduce risk of postpartum hemorrhage; sensitize cells to the oxytocin; or increase OXTR signaling. In some embodiments, the OXTR chaperone is administered in an amount effective to reduce risk of adverse events, such as cesarean section, uterine atony, or post-partum hemorrhage. In some embodiments, the OXTR chaperone is administered in an amount effective to modulate social behavior. In some embodiments, the OXTR chaperone is administered in an amount effective to modulate lactation. In some embodiments, the subject has oxytocin insensitivity. In some embodiments, the subject has loss-of-function (variants that would normally impair OXTR trafficking and decrease oxytocin response) OXTR genetic variants (e.g., V281M, E339K). In some embodiments, the subject has or is suspected of having autism spectrum disorder. In some embodiments, the subject has or is suspected of having a psychiatric condition. In some embodiments, the subject has pain or is in need of pain relief.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 depicts how small molecule modulators of oxytocin receptor (OXTR) increase the number of OXTRs on the surface of human myometrial cells. 9 commercially-available small molecule agonists and antagonists of OXTR and vasopressin receptor and found three that significantly increased the number of OXTRs on the cell surface (N=3, P<0.05). These experiments were performed in the hTERT-immortalized human myometrial cell line, which was modified to introduce an N-terminal HA tag upstream of OXTR. Cells were incubated overnight with 10 μM of the indicated compound or vehicle control. OXTR was quantified by performing flow cytometry with a fluorescent antibody against HA. TCOT=TC-OT 39 ((2S)—N-[[4-[(4,10-dihydro-1-methylpyrazolo[3,4-b][1,5]benzodiazepin-5(1H)-yl)carbonyl]-2-methylphenyl]methyl]-2-[(hexahydro-4-methyl-1H-1,4-diazepin-1-yl)thioxomethyl]-1-pyrrolidinecarboxamide), partial OXTR agonist. CON=conivaptan (N-[4-(2-methyl-4,5-dihydro-3H-imidazo[4,5-d][1]benzazepine-6-carbonyl)phenyl]-2-phenylbenzamide; also known as YM087), vasopressin inhibitor. TASP=TASP 0390325 (2-(3-Chloro-4-fluorophenyl)-N-(1-methylethyl)-6-[3-(4-morpholinyl)propoxy]-4-oxo-pyrido[2,3-d]pyrimidine-3(4H)-acetamide hydrochloride), vasopressin inhibitor. WAY=WAY 267464 (4-(3,5-Dihydroxybenzyl)-N-(2-methyl-4-[(1-methyl-4,10-dihydropyrazolo[3,4-b][1,5]benzodiazepin-5(1H)-yl)carbonyl]benzyl)piperazine-1-carboxamide), OXTR agonist. TOL=tolvaptan (N-(4-{[(5R)-7-Chloro-5-hydroxy-2,3,4,5-tetrahydro-1H-1-benzazepin-1-yl]carbonyl}-3-methylphenyl)-2-methylbenzamide; also known as OPC41061), vasopressin inhibitor. OPC=OPC 21268 (N-[3-[4-[[4-(3,4-dihydro-2-oxo-1(2H)-quinolinyl)-1-piperidinyl]carbonyl]phenoxy]propyl]-acetamide), vasopressin inhibitor. SSR=SSR 149415 ((2S,4R)-1-[(3R)-5-Chloro-1-[(2,4-dimethoxyphenyl)sulfonyl]-2,3-dihydro-3-(2-methoxyphenyl)-2-oxo-1H-indol-3-yl]-4-hydroxy-N,N-dimethyl-2-pyrrolidinecarboxamide), vasopressin inhibitor. SR=SR49059 ((2S)-1-[[(2R,3S)-5-Chloro-3-(2-chlorophenyl)-1-[(3,4-dimethoxyphenyl)sulfonyl]-2,3-dihydro-3-hydroxy-1H-indol-2-yl]carbonyl]-2-pyrrolidinecarboxamide), vasopressin inhibitor. L371=L371,257 (1-[1-[4-(1-acetylpiperidin-4-yl)oxy-2-methoxybenzoyl]piperidin-4-yl]-4H-3,1-benzoxazin-2-one), OXTR antagonist.

FIG. 2 depicts how SR49059 increases oxytocin response in human myometrial cells. Myometrial cells were incubated with 10 μM SR49059 overnight. The cells' response to oxytocin was assayed. Oxytocin response was quantified by performing IP1 accumulation assays, which measure oxytocin signaling through the Gq-phospholipase C pathway. SR49059 treatment increased maximal oxytocin response by 12% (N=3, P<0.05).

FIG. 3 depicts how SR49059 improves cell surface localization of V281M OXTR. The effect of SR49059 on cell surface localization of a variant OXTR that is normally retained inside the cell was studied. The V281M genetic variant is most prevalent in the Swedish population, where it is present in 7-8 out of every 1000 individuals. These experiments were performed in HEK293 cells stably transfected with wild type (WT) OXTR-GFP or with variant (V281M) OXTR-GFP. Overnight incubation of cells with 10 μM SR49059 improved cells' surface localization of V281M OXTR.

FIG. 4 depicts how SR49059 rescues function in cells transfected with V281M OXTR. HEK293T cells were transfected with wild type (WT) and V281M OXTR and incubated overnight with 10 μM SR49059 or vehicle control. The cellular response to oxytocin was measured as in FIG. 2. SR49059 treatment increased maximal response to oxytocin by 16% in V281M-transfected cells (N=3, P<0.05). After treatment, response was indistinguishable from the wild type response.

FIG. 5A depicts how SR49059 and L371,257 increase cell surface abundance of variant oxytocin receptor in HEK293T cells. The effects of compounds on V281M OXTR cell surface abundance in HEK293T cells are plotted relative to cell surface OXTR abundance in cells transfected with WT OXTR and treated with vehicle. *p<0.05 compared with vehicle-treated cells by one-way ANOVA with Dunnett's multiple comparisons test. Data shown are mean and standard error from N=3 independent trials. OXTR, oxytocin receptor.

FIG. 5B depicts SR49059 and L371,257 increase cell surface abundance of wild-type oxytocin receptor in HEK293T cells. The effects of compounds on WT OXTR cell surface abundance are shown relative to that in vehicle-treated cells. **p<0.0055 compared with vehicle by one-sample t test (a corrected for multiple comparisons by Bonferroni method). Data shown are mean and standard error from N=3 independent trials. OXTR, oxytocin receptor.

FIG. 6A-6C depict how SR49059 and L371,257 rescue trafficking and functional defects of V281M OXTR.

FIG. 6A depicts subcellular localization of OXTR and ER marker PD1 (top) or Golgi marker Golgin-97 (bottom) in HEK293 cells stably transfected with WT OXTR-GFP and V281M OXTR-GFP. Results shown are representative from three independent trials.

FIG. 6B depicts quantitation of colocalization by Pearson's r of OXTR and PD1 or Golgin-97 of images like the examples in FIG. 6A. Each point represents an individual image. ****p<0.0001 by one-way ANOVA with Dunnett's multiple comparisons test.

FIG. 6C depicts oxytocin-induced IP1 production in HEK293T cells transfected with WT or V281M OXTR and treated with vehicle or SR49059. Data are shown as mean and standard error from N=5 independent trials. ****p<0.0001 compared with V281M+vehicle by sum-of-squares F test. OXTR, oxytocin receptor.

FIG. 7A depicts the effects of V281M mutation and chaperone treatment on basal concentration by depicting the IP1 concentration in unstimulated cells (no oxytocin treatment). *P<0.05, **P<0.005, ****P<0.0001 by one-way ANOVA with Šidà\acute{k}'s multiple comparisons test; ns, not significant. Data shown are mean and standard error from N=5 independent trials.

FIG. 7B depicts the effects of V281M mutation and chaperone treatment on maximal concentration by IP1 concentration in cells treated with 10 μM oxytocin. *P<0.05, **P<0.005, ****P<0.0001 by one-way ANOVA with Šidák's multiple comparisons test; ns, not significant. Data shown are mean and standard error from N=5 independent trials.

FIG. 8A-D depicts how SR49059 and L371,257 increase OXTR trafficking and oxytocin response in immortalized human myometrial cells.

FIG. 8A depicts the effect of compounds on cell surface localization of OXTR in hTERT-HM cells. Data shown are mean and standard error from N=3 independent trials. *p<0.05, **p<0.01 by one-way ANOVA with Dunnett's multiple comparisons test. OXTR, oxytocin receptor.

FIG. 8B depicts the change in surface OXTR on hTERT-HM cells after incubation with SR49059 or L371,257 for the indicated time points. Error bars show standard error for N=5 (SR49059) and N=3 (L371,257). OXTR, oxytocin receptor.

FIG. 8C depicts the change in surface OXTR after 12-h incubation with SR49059, L371,257, cycloheximide, and brefeldin A as indicated. Bars with “a” are statistically different from vehicle-treated cells at p<0.001 (one-sample t test). *p<0.05 by one-way ANOVA with Šidák's multiple comparisons test; ns, not significant. OXTR, oxytocin receptor.

FIG. 8D depicts oxytocin-induced IP1 production in hTERT-HM cells treated with SR49059 or L371,257. Data are shown as mean and standard error from N=5 independent trials. ****p<0.0001 compared with vehicle-treated cells by sum-of-squares F test. OXTR, oxytocin receptor.

FIG. 9A-F depict how SR49059 and L371,257 increase oxytocin-induced IP1 production in primary human myometrial cells.

FIG. 9A depicts oxytocin-induced IP1 production in primary human myometrial samples from term nonlaboring sample TNL 1. Error bars show standard error from N=4 technical replicates. p-values from comparison of curve Emax (sum-of-squares F test).

FIG. 9B depicts oxytocin-induced IP1 production in primary human myometrial samples from term nonlaboring sample TNL 2. Error bars show standard error from N=4 technical replicates. p-values from comparison of curve Emax (sum-of-squares F test).

FIG. 9C depicts oxytocin-induced IP1 production in primary human myometrial samples from term nonlaboring sample TNL 3. Error bars show standard error from N=4 technical replicates. p-values from comparison of curve Emax (sum-of-squares F test).

FIG. 9D depicts oxytocin-induced IP1 production in primary human myometrial samples from term nonlaboring sample TNL 4. Error bars show standard error from N=4 technical replicates. p-values from comparison of curve Emax (sum-of-squares F test).

FIG. 9E depicts oxytocin-induced IP1 production in primary human myometrial samples from term nonlaboring sample TNL 5. Error bars show standard error from N=4 technical replicates. p-values from comparison of curve Emax (sum-of-squares F test).

FIG. 9F depicts Emax from N=5 primary cell samples. p values from Friedman ANOVA with Dunn's multiple comparisons test.

### DETAILED DESCRIPTION OF THE INVENTION

The present disclosure is based, at least in part, on the discovery of pharmacological chaperones to improve trafficking of the oxytocin receptor and enhance clinical response to oxytocin. As shown herein, pharmacological chaperones for the oxytocin receptor sensitized cells to oxytocin treatment (see Example 1).

Background: Oxytocin is a nine-amino acid hormone that is produced naturally in the body, as well as administered as a drug. The release of endogenous oxytocin from the posterior pituitary gland modulates social behavior, lactation, and uterine contractions. Clinicians commonly use synthetic oxytocin to increase uterine contractions during childbirth: this method is used to induce and augment labor, as well as to prevent postpartum hemorrhage, in almost all patients who give birth in the United States. Herein is described a method to sensitize cells to the oxytocin, thereby enhancing oxytocin response. In order to respond to oxytocin, cells must display oxytocin receptors (OXTRs) on their plasma membrane. However, a considerable store of OXTRs (˜80%) are located inside the cell in the endoplasmic reticulum and Golgi body.

The technology is validated in various cell cultures, including uterine smooth muscle cells, using recombinant versions of OXTR.

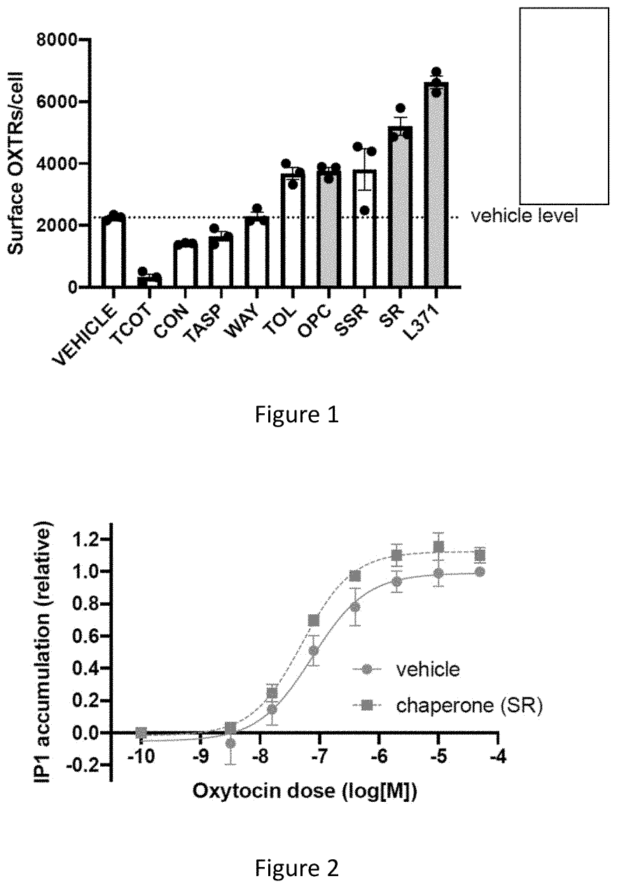

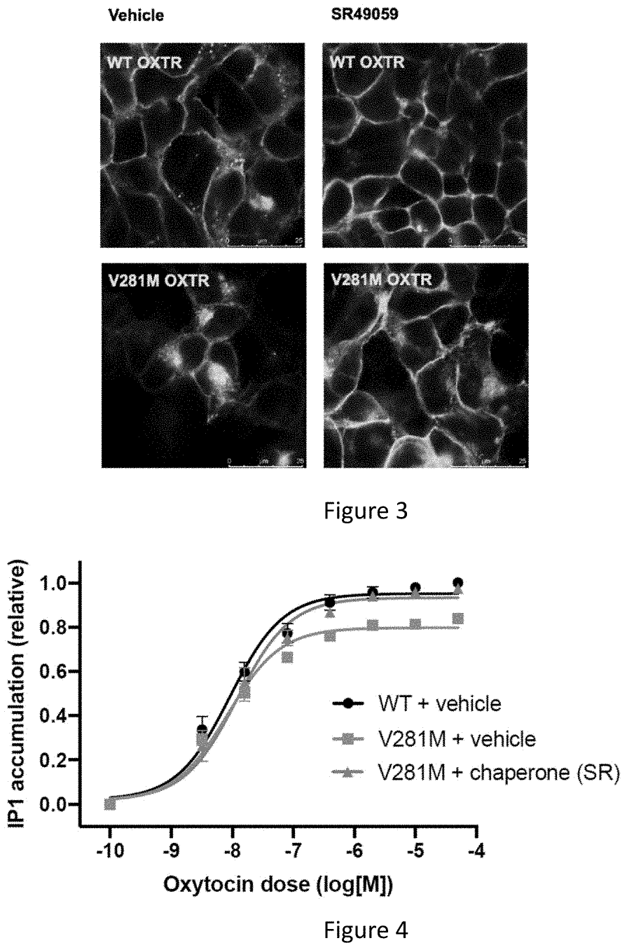

Overview: It is shown that small molecules that bind to functional sites in OXTR act as pharmacological chaperones for OXTR, increasing the trafficking of the receptors from intracellular stores to the cell surface (FIG. 1). Long-term treatment (>6 hours) with these drugs increases the number of OXTRs on the cell surface and increases OXTR signaling in uterine smooth muscle cells (FIG. 2). This treatment also improves OXTR trafficking and oxytocin response for OXTR genetic variants (FIG. 3). These variants normally impair OXTR trafficking and decrease oxytocin response; treatment reverses this effect (FIG. 4). This effect has been demonstrated with commercially-available antagonists for OXTR and vasopressin receptor. However, compounds that bind to other sites in OXTR could be identified or designed that can recapitulate these effects.

Oxytocin is administered during childbirth to almost every single patient—millions of people per year—for labor induction, labor augmentation, and/or prevention of post-partum hemorrhage. Furthermore, oxytocin is used as an experimental therapy for autism spectrum disorder, other psychiatric conditions, and pain. However, response to oxytocin varies widely between individuals, with many patients failing to respond. Failure to respond to oxytocin during childbirth can lead to emergency Cesarean sections, uterine atony, and post-partum hemorrhage, which are major causes of maternal morbidity and mortality.

Treatment with an oxytocin receptor chaperone was shown to enhance uterine cells' ability to respond to oxytocin. Use of an oxytocin receptor chaperone could prevent the adverse events associated with oxytocin non-response, and could be broadly applicable to any scenario in which exogenous oxytocin is administered. The disclosed technology could also enhance the endogenous actions of oxytocin in the body, including the modulation of social behavior and lactation.

Technology: The present disclosure describes a method of treating oxytocin hyposensitivity by using a class of compounds called pharmacologic chaperones. These drugs (such as SR49059) act by promoting oxytocin receptor (OXTR) folding and localization to the plasma membrane, so that more of the receptor can bind oxytocin and elicit a cellular response. In individuals with mutated OXTR, such as V281M and E339K, these chaperones can restore normal OXTR signaling by eliminating the intracellular accumulation of OXTR and bringing more functional receptors to the cell surface. Even in individuals with wild-type OXTR, these chaperones can further enhance the surface localization of OXTR and thus increase the overall sensitivity of cells to the oxytocin ligand.

These pharmacologic chaperones can rescue the folding of a misfolded mutant or wild-type OXTR protein. The chaperones can also enhance translation and/or trafficking of mutant or wild-type OXTR.

Oxytocin is secreted from the posterior pituitary gland. Endogenous oxytocin promotes social bonding, lactation, and uterine contractions during labor. Synthetic oxytocin is given to women to induce or augment labor, but the doses required differ greatly among individuals. Under-response to oxytocin can lead to a variety of complications and is a major contributor to morbidity/mortality during childbirth. Therapeutic oxytocin is also being investigated as a treatment for autism spectrum disorders. Administering oxytocin may be beneficial to people with oxytocin deficiency, but not to those with low sensitivity to the hormone. OXTR is a GPCR, and it is the only known endogenous receptor for oxytocin. Oxytocin sensitivity can be attributed to the allelic variations in OXTR, where mutations such as V281M are clinically associated with dramatically reduced physiological response to oxytocin. One reason why oxytocin response is so poor in OXTR V281M individuals is that the receptor appears to be trapped inside ER and Golgi, where it is unable to bind to the oxytocin ligand at the cell surface.

To elicit a robust oxytocin response, the current method is to administer a large therapeutic dose of synthetic oxytocin (e.g., Pitocin®). However, the effects of exogenous oxytocin vary greatly among individuals. Also, for patients with certain genetic variants of OXTR, giving exogenous oxytocin will not elicit a physiological response.

The present disclosure addresses depressed oxytocin response by normalizing the underlying defects in the receptor, one of which is mislocalization or under-localization of OXTR to the cell surface, where it can interact with oxytocin. Using pharmacologic chaperones, the inventors discovered that they can significantly enhance the surface localization of both wild-type and mutant OXTRs. Moreover, improved receptor surface localization is accompanied by an increased physiological response to oxytocin. In effect, the disclosed invention greatly sensitizes relevant cell types to the oxytocin ligand.

The present disclosure provides for:

i. A treatment with the potential to transform the utilization of synthetic oxytocin, which is beset with many efficacy issues. The disclosed chaperons increase functional OXTR cell surface display or expression as a way to sensitize cells to oxytocin.

ii. This treatment can greatly improve the effects of endogenous oxytocin, as well as the patient's response to synthetic oxytocin.

iii. Can promote oxytocin response in OXTR mutant individuals, for whom no treatment currently exists.

iv. Drug compounds under analysis (e.g., SR49059) are commercially available.

Benefits can include:

i. Enhancing the efficacy of oxytocin therapy by acting as an adjuvant.

ii. Effectively treat a cohort of patients with congenital OXTR mutations by restoring their sensitivity to oxytocin. These patients may not have been treated by exogenous oxytocin alone, because their issue is not oxytocin deficiency, but oxytocin insensitivity.

iii. Enhancing the sensitivity of wild-type OXTR to endogenously produced oxytocin, and elicit similar physiological responses as if exogenous oxytocin were given. Therefore, these pharmacologic chaperones could potentially replace some oxytocin therapy, especially in countries without the cold storage infrastructure to maintain a stable oxytocin supply.

Pharmaceutical OXTR Chaperone: Oxytocin Receptor (OXTR) or Vasopressin Receptor Binding Agents

As described herein, OXTR located inside the cell and not on the cell surface has been implicated in various diseases, disorders, and conditions. As such, modulation of OXTR (e.g., modulation of OXTR or vasopressin) display can be used for the treatment of such conditions. A pharmaceutical chaperone can increase the number of OXTRs on the cell surface. A pharmacologic chaperone can rescue the folding of a misfolded mutant or wild-type OXTR protein and can also enhance translation and/or trafficking of mutant or wild-type OXTR. An OXTR modulation agent can modulate the display or expression of OXTR on the cells, modulating the quantity or number of cells that express OXTR, or modulating the quality of the OXTR expressing cells. Here, agonists and antagonists for OXTR and vasopressin receptor were used as a pharmaceutical chaperone to OXTR.

OXTR binding agents can be any composition or method that can modulate OXTR expression or display on cells. For example, an OXTR binding agent can be an activator, an inhibitor, an agonist, or an antagonist. As another example, the OXTR binding can be the result of gene editing.

An OXTR binding agent can be an OXTR antibody (e.g., a monoclonal antibody to OXTR).

OXTR Chaperone/Binding Agents

One aspect of the present disclosure provides for targeting of OXTR and vasopressin receptor.

As described herein, binding agents can be antibodies, fusion proteins, small molecules and can induce OXTR cell surface display.

As another example, the binding agent can be a fusion protein. As another example, a binding agent can be an inhibitory protein that antagonizes OXTR or vasopressin receptors.

Examples of OXTR chaperones or binding agents are described herein. chaperone or binding agents can be, analogs or derivatives of: tolvaptan (N-(4-{[(5R)-7-Chloro-5-hydroxy-2,3,4,5-tetrahydro-1H-1-benzazepin-1-yl]carbonyl}-3-methylphenyl)-2-methylbenzamide; also known as OPC41061), a vasopressin inhibitor, such as tolvaptan, OPC 21268 (N-[3-[4-[[4-(3,4-dihydro-2-oxo-1(2H)-quinolinyl)-1-piperidinyl]carbonyl]phenoxy]propyl]-acetamide), SSR 149415 ((2S,4R)-1-[(3R)-5-Chloro-1-[(2,4-dimethoxyphenyl)sulfonyl]-2,3-dihydro-3-(2-methoxyphenyl)-2-oxo-1H-indol-3-yl]-4-hydroxy-N,N-dimethyl-2-pyrrolidinecarboxamide), SR49059 ((2S)-1-[[(2R,3S)-5-Chloro-3-(2-chlorophenyl)-1-[(3,4-dimethoxyphenyl)sulfonyl]-2,3-dihydro-3-hydroxy-1H-indol-2-yl]carbonyl]-2-pyrrolidinecarboxamide) or an OXTR antagonist, such as L371,257 (1-[1-[4-(1-acetylpiperidin-4-yl)oxy-2-methoxybenzoyl]piperidin-4-yl]-4H-3,1-benzoxazin-2-one).

Substituent (e.g., R) groups can be added, removed, or optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxyl; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “carbonyl”, as used herein, unless otherwise indicated, can include a functional group consisting of a carbon atom double-bonded to an oxygen atom (C═O). The “carbonyl” can be optionally substituted.

The term “alkenyl”, as used herein, unless otherwise indicated, can include alkyl moieties having at least one carbon-carbon double bond wherein alkyl is as defined above and including E and Z isomers of the alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated.

The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O— cyclobutyl, —O-cyclopentyl, —O-cyclohexyl, —O-cycloheptyl, —O-cyclooctyl, —O— cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O— CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH₂— cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S, and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “hetreocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example, water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1-methylene-bis-(2-hydroxy-3-naphthoate)) salts. A pharmaceutically acceptable salt may involve the inclusion of another molecule such as an acetate ion, a succinate ion, or another counterion. The counterion may be any organic or inorganic moiety that stabilizes the charge on the parent compound. Furthermore, a pharmaceutically acceptable salt may have more than one charged atom in its structure. In instances where multiple charged atoms are part of the pharmaceutically acceptable salt, the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:

(i) By disabling repressors. The gene is expressed because an inducer binds to the repressor. The binding of the inducer to the repressor prevents the repressor from binding to the operator. RNA polymerase can then begin to transcribe operon genes.

(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

So-called conservative exchanges can be carried out in which the amino acid which is replaced has a similar property as the original amino acid, for example, the exchange of Glu by Asp, Gln by Asn, Val by lie, Leu by lie, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63(% formamide)−(600/I). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating, preventing, or reversing a disease, disorder, or condition associated with OXTR in a subject in need of administration of a therapeutically effective amount of an OXTR chaperone, so as to increase the number of OXTRs on the surface of a cell or increase oxytocin response.

Methods described herein are generally performed on a subject in need thereof. A subject in need of the therapeutic methods described herein can be a subject having, diagnosed with, suspected of having, or at risk for developing a disease, disorder, or condition associated with OXTR (e.g., a disease, disorder, or condition associated with reduced cell-surface OXTR, such as variants, e.g., V281M). A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of an OXTR chaperone is, for example, an amount that would cause the desired therapeutic effect in a subject while minimizing undesired side effects. In various embodiments, an effective amount of an OXTR chaperone described herein can substantially increase cell surface OXTR.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of an OXTR chaperone can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to increase cell surface OXTR.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of an OXTR chaperone can occur as a single event or over a time course of treatment. For example, an OXTR chaperone can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for a disease, disorder, or condition associated with OXTR or reduced OXTR on surface of cells.

An OXTR chaperone can be administered simultaneously or sequentially with another agent, such as an antibiotic, an anti-inflammatory, or another agent. For example, an OXTR chaperone can be administered simultaneously with another agent, such as an antibiotic or an anti-inflammatory. Simultaneous administration can occur through administration of separate compositions, each containing one or more of an OXTR chaperone, an antibiotic, an anti-inflammatory, or another agent. Simultaneous administration can occur through administration of one composition containing two or more of an OXTR chaperone, an antibiotic, an anti-inflammatory, or another agent. An OXTR chaperone can be administered sequentially with an antibiotic, an anti-inflammatory, or another agent. For example, an OXTR chaperone can be administered before or after administration of an antibiotic, an anti-inflammatory, or another agent.

Cell Therapy

Cells treated with an OXTR chaperone according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation has gained significant interest by researchers as a potential therapeutic strategy for a wide range of diseases, in particular for degenerative and immunogenic pathologies.

Allogeneic cell therapy or allogenic transplantation uses donor cells from a different subject than the recipient of the cells. A benefit of an allogenic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

Screening

Also provided are screening methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlog P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8A to about 15A.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to OXTR chaperone(s), assays or components of the assays to test the chaperones, or cells. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice.

However, those of skill in the art should, in light of the present disclosure, appreciate that many changes can be made in the specific embodiments that are disclosed and still obtain a like or similar result without departing from the spirit and scope of the present disclosure.

### Example 1: Pharmacological Chaperones to Improve Trafficking of the Oxytocin Receptor and Enhance Clinical Response to Oxytocin

Oxytocin is a nine-amino acid hormone that is produced naturally in the body, as well as administered as a drug. Release of endogenous oxytocin from the posterior pituitary gland modulates social behavior, lactation, and uterine contractions. Clinicians commonly use synthetic oxytocin to increase uterine contractions during childbirth: this method is used to induce and augment labor, as well as to prevent postpartum hemorrhage, in almost all patients who give birth in the United States.

Oxytocin is administered during childbirth to almost every single patient—millions of people per year—for labor induction, labor augmentation, and/or prevention of post-partum hemorrhage. Furthermore, oxytocin is used as an experimental therapy for autism spectrum disorder, other psychiatric conditions, and pain. However, response to oxytocin varies widely between individuals, with many patients failing to respond. Failure to respond to oxytocin during childbirth can lead to emergency Cesarean sections, uterine atony, and post-partum hemorrhage, which are major causes of maternal morbidity and mortality. Thus, identifying patients at risk for poor oxytocin response and developing strategies to enhance oxytocin response would have clinical utility.

Genetic variants in the oxytocin receptor (OXTR) were hypothesized to impair trafficking of the receptor to the cell surface, thus decreasing oxytocin response. It was investigated whether pharmacological chaperones could mobilized OXTR to the cell membrane and enhance cellular response to oxytocin.

Experiments were performed in HEK293T cells transfected with wild type (WT) and variant OXTR and in hTERT-immortalized human myometrial (hTERT-HM) cells. Missense genetic variants for study were selected from the gnomAD database based on prevalence and a functional screen. OXTR was tagged with an N-terminal HA tag to enable detection of cell surface and total OXTR by quantitative flow cytometry. Oxytocin response was measured by using fluorescence-based calcium flux assays and inositol monophosphate (IP1) accumulation assays.

Here, a method is described to sensitize cells to the oxytocin, thereby enhancing the oxytocin response. In order to respond to oxytocin, cells must display oxytocin receptor (OXTRs) on their plasma membrane. However, a considerable store of OXTRs (˜80%) are located inside the cell in the endoplasmic reticulum and Golgi body. Small molecules that bind to functional sites in OXTR act as pharmacological chaperones for OXTR, increasing the trafficking of the receptors from intracellular stores to the cell surface (FIG. 1). Long-term treatment (>6 hours) with these drugs increases the number of OXTRs on the cell surface and increases OXTR signaling in uterine smooth muscle cells (FIG. 2). This treatment also improves OXTR trafficking and oxytocin response for OXTR genetic variants (FIG. 3). These variants normally impair OXTR trafficking and decrease oxytocin response, treatment reverses this effect (FIG. 4). Thus far, this effect was demonstrated with commercially available antagonists for OXTR and vasopressin receptors.

Two of the four variants analyzed, V281M and E339K, impair receptor trafficking from the endoplasmic reticulum and Golgi body to the cell surface. V281M and E339K decrease cell surface OXTR localization by 49% and 36%, respectively, compared to WT OXTR. Accordingly, these variants reduce maximal oxytocin signaling by 23% and 22%. Next, it was investigated whether small molecule modulators of OXTR could act as pharmacological chaperones, increasing the trafficking of OXTR from intracellular stores to the cell membrane. Overnight (16 h) treatment with three modulators increases the cell surface localization of V281M OXTR to above WT levels. These same modulators increase cell surface localization of the WT OXTR in hTERT-HM cells by three-fold. Finally, pre-treatment with one modulator increases oxytocin-induced IP1 accumulation in hTERT-HM cells by 12%.

Genetic variants present in the human population impair trafficking of OXTR, which can decrease oxytocin response. Pharmacological chaperones can be a useful treatment strategy for patients predicted to have poor oxytocin response due to genetic or other factors.

Treatment with an oxytocin receptor chaperone enhances uterine cells' ability to respond to oxytocin. The use of an oxytocin receptor chaperone could prevent the adverse events associated with oxytocin non-response, and could be broadly applicable to any scenario in which exogenous oxytocin is administered.

This invention could also enhance the endogenous actions of oxytocin in the body, including the modulation of social behavior and lactation.

### Example 2: Small-Molecule OXTR Antagonists Rescue Trafficking and Functional Defects of V281M OXTR

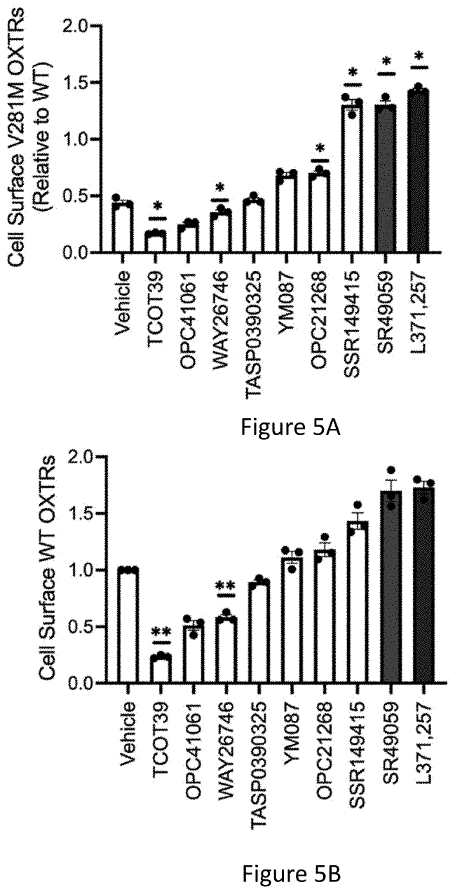

To identify molecules with pharmacoperone activity, nine commercially available small-molecule ligands that bind the oxytocin and/or vasopressin receptor were screened (Table 1). Both agonists and antagonists with varying reported affinities for OXTR were included. Given their high calculated lipophilicity values (log P>2.8), all tested compounds were predicted to permeate the cell membrane. HEK293T cells were transiently transfected with V281M OXTR tagged with hemagglutinin and GFP (HA-OXTR-GFP), treated them with 10 μM of each candidate drug for 16 h, then performed quantitative flow cytometry to measure cell surface OXTR. Vehicle-treated cells transfected with V281M HA-OXTR-GFP had 57% fewer OXTRs on the cell surface than vehicle-treated cells transfected with WT HA-OXTR-GFP (FIG. 5A). The two known OXTR agonists—TCOT39 and WAY26746—decreased cell surface V281M OXTR, likely due to β-arrestin-induced internalization after OXTR activation. TASP0390325, which has extremely low affinity for OXTR, had no effect on cell surface V281M OXTR. In contrast, three antagonists—SSR1494155, SR49059, and L371,257—increased cell surface V281M OXTR to 130 to 143% of WT levels (FIG. 5A-B, p=0.019, 0.012, 0.004, respectively).

TABLE 1
Candidate pharmacoperones screened for
effects on OXTR cell surface expression

| Drug | Primary action |
|---|---|
| TCOT39 ((2S)-N-[[4-[(4,10-dihydro-1- | Selective OXTR |
| methylpyrazolo[3,4- | partial agonist |
| b][1,5]benzodiazepin-5(1H)- | |
| yl)carbonyl]-2-methylphenyl]methyl]-2- | |
| [(hexahydro-4-methyl-1H-1,4- | |
| diazepin-1-yl)thioxomethyl]-1- | |
| pyrrolidinecarboxamide) | |
| OPC41061 (N-(4-{[(5R)-7-Chloro-5- | AVPR2 antagonist; |
| hydroxy-2,3,4,5-tetrahydro-1H-1- | unknown OXTR affinity |
| benzazepin-1-yl]carbonyl}-3- | |
| methylphenyl)-2-methylbenzamide; | |
| also known as tolvaptan) | |
| WAY26746 (4-(3,5-Dihydroxybenzyl)- | Selective OXTR agonist |
| N-(2-methyl-4-[(1-methyl-4,10- | |
| dihydropyrazolo[3,4- | |
| b][1,5]benzodiazepin-5(1H)- | |
| yl)carbonyl]benzyl)piperazine-1- | |
| carboxamide) | |
| TASP0390325 (2-(3-Chloro-4- | Selective AVPR1A |
| fluorophenyl)-N-(1-methylethyl)-6-[3- | antagonist; low |
| (4-morpholinyl)propoxy]-4-oxo- | OXTR affinity |
| pyrido[2,3-d]pyrimidine-3(4H)- | |
| acetamide hydrochloride) | |
| YM087 (N-[4-(2-methyl-4,5-dihydro- | AVPR1A/2 antagonist; |
| 3H-imidazo[4,5-d][1]benzazepine-6- | medium OXTR affinity |
| carbonyl)phenyl]-2-phenylbenzamide; | |
| also known as conivaptan) | |
| OPC21268 (N-[3-[4-[[4-(3,4-dihydro-2- | AVPR1 antagonist; |
| oxo-1(2H)-quinolinyl)-1- | medium OXTR affinity |
| piperidinyl]carbonyl]phenoxy]propyl]- | |
| acetamide) | |
| SSR149415 ((2S,4R)-1-[(3R)-5- | AVPR1B antagonist; |
| Chloro-1-[(2,4- | medium OXTR affinity |
| dimethoxyphenyl)sulfonyl]-2,3- | |
| dihydro-3-(2-methoxyphenyl)-2-oxo- | |
| 1H-indol-3-yl]-4-hydroxy-N,N- | |
| dimethyl-2-pyrrolidinecarboxamide) | |
| SR49059 ((2S)-1-[[(2R,3S)-5-Chloro- | AVPR1A antagonist; |
| 3-(2-chlorophenyl)-1-[(3,4- | medium OXTR affinity |
| dimethoxyphenyl)sulfonyl]-2,3- | |
| dihydro-3-hydroxy-1H-indol-2- | |
| yl]carbonyl]-2-pyrrolidinecarboxamide) | |
| L371,257 (1-[1-[4-(1-acetylpiperidin-4- | Selective OXTR antagonist |
| yl)oxy-2-methoxybenzoyl]piperidin-4- | |
| yl]-4H-3,1-benzoxazin-2-one) | |

Next the effect of these same compounds was examined in cells transfected with WT HA-OXTR-GFP. Most had similar effects as they did on V281M OXTR (FIG. 5B). Cells treated with two compounds-SR49059 and L371,257-had 170% more surface WT OXTR than vehicle-treated cells (FIG. 5B). Therefore, these two compounds were focused on in further experiments.

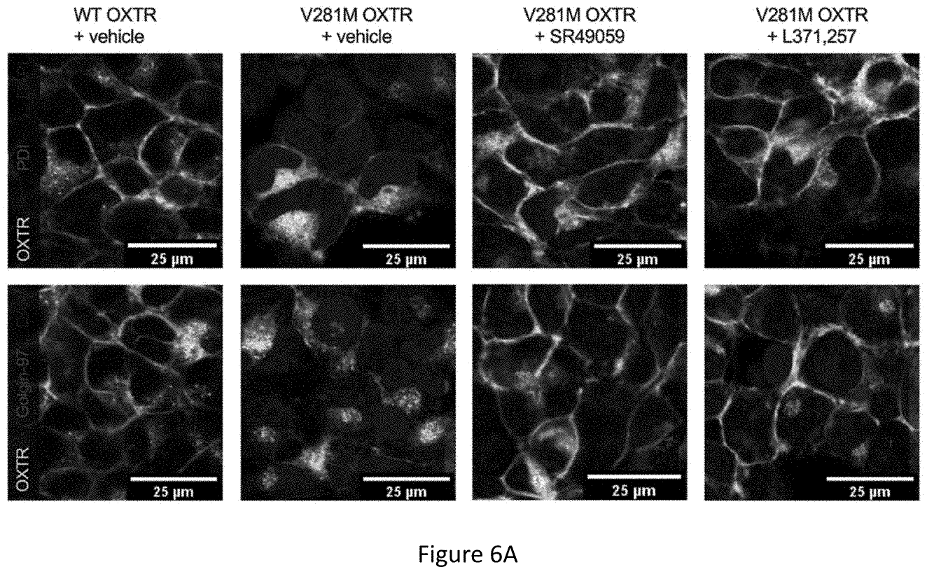

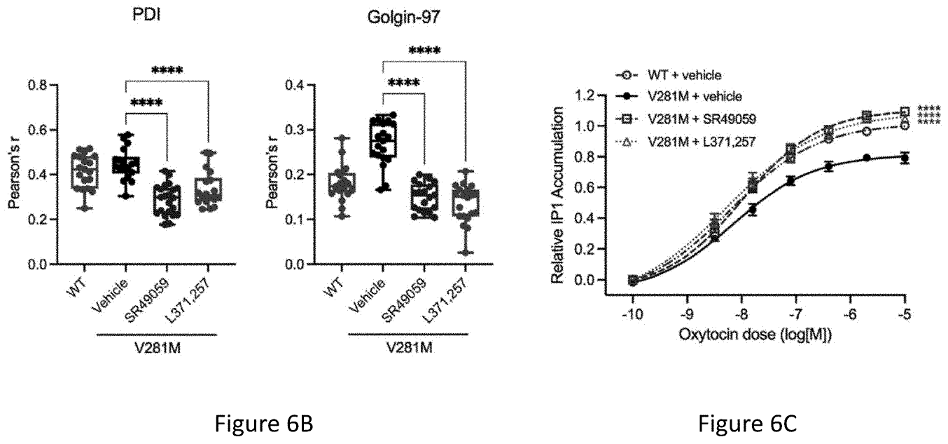

Next confocal microscopy was used to determine the effect of SR49059 and L371,257 on subcellular localization of V281M OXTR-GFP in stably transfected HEK293 cells. In vehicle-treated cells, V281M OXTR-GFP accumulated intracellularly (FIG. 6A). Costaining revealed that intracellular V281M OXTR-GFP colocalized with markers for the endoplasmic reticulum (ER) (protein disulfide isomerase, PDI) and Golgi (golgin-97). Treatment with 10 μM SR49059 or L371,257 decreased colocalization of V281M OXTR-GFP with both PDI and golgin-97 (p<0.0001). SR49059 and L371,257 allowed V281M OXTR-GFP to traffic through the ER and Golgi to reach the cell membrane, consistent with flow cytometry results.

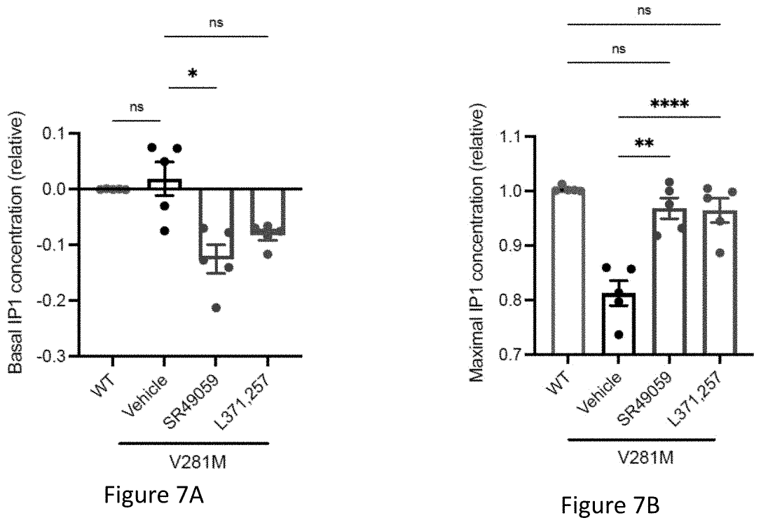

The enhanced V281M OXTR cell surface localization was hypothesized in SR49059- and L371,257-treated cells to lead to increased oxytocin response. To test this idea, HEK293T cells were transiently transfected with V281M OXTR, incubated them with oxytocin, and quantified inositol monophosphate (IP1), a downstream product of signaling through the Gq-phospholipase C-inositol triphosphate pathway. At baseline (no oxytocin stimulation), IP1 concentration was similar in cells transfected with WT OXTR and V281M OXTR (FIG. 7A). Treatment with SR49059 slightly decreased basal IP1 concentrations in V281M OXTR-transfected cells (p=0.020); treatment with L371,257 had a similar, but not statistically significant, effect (p=0.057, FIG. 7A). Stimulation with oxytocin resulted in a dose-dependent increase in IP1 concentration in all cells (FIG. 6C). Consistent with previous results, maximal oxytocin-induced IP1 production in vehicle-treated V281M OXTR-expressing cells was 80% of that in WT OXTR-expressing cells (FIG. 6C). However, treatment with SR49059 and L371,257 abolished this difference (FIG. 6C); IP1 concentrations in oxytocin-stimulated cells were similar in V281M OXTR-expressing cells treated with SR49059 or L371,257 and WT OXTR-expressing cells treated with vehicle control (FIG. 7B). Taken together, these data showed that SR49059 and L371,257 restored both OXTR cell surface localization and oxytocin response in V281M OXTR-expressing cells.

### Example 3: SR49059 and L371,257 Increase WT OXTR Cell Surface Localization in Immortalized Human Myometrial Cells

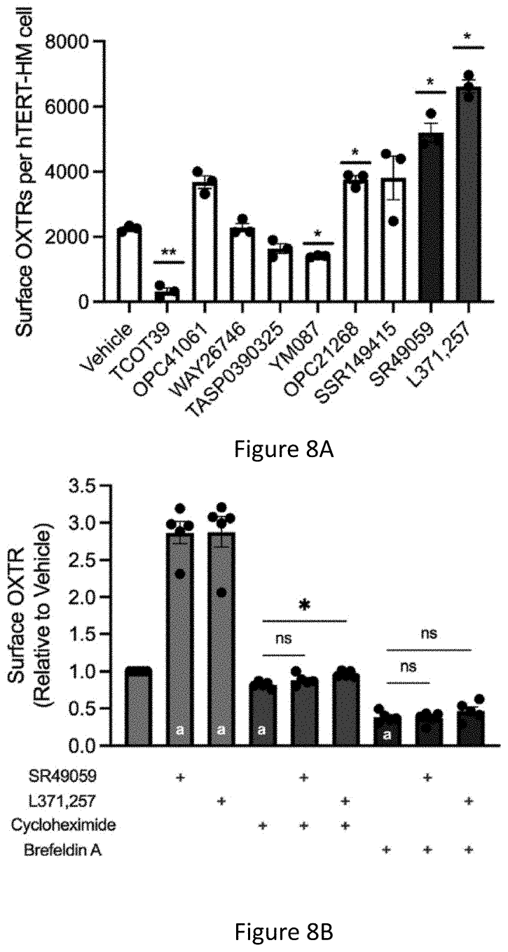

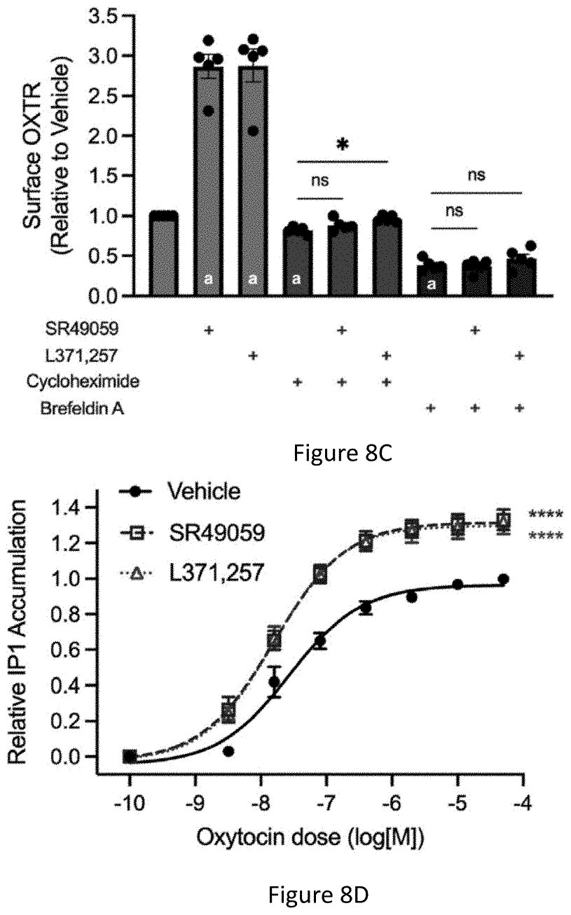

Given the robust effects of SR49059 and L371,257 in HEK293T cells, these compounds were hypothesized to enhance trafficking of WT OXTR endogenously expressed in hTERT-immortalized human myometrial (hTERT-HM) cells, which endogenously express WT OXTR. Because specific antibodies to OXTR are not commercially available, CRISPR-Cas9 was used to introduce an HA tag at the N-terminus of OXTR in the hTERT-HM cell line. Then quantitative flow cytometry was performed to assess the effect of the nine candidate pharmacoperones on cell surface HA-OXTR (16 h treatment, 10 μM). As in HEK293T cells, the OXTR partial agonist TCOT39 decreased surface abundance of OXTRs (p=0.009). However, the OXTR agonist WAY26746, which had decreased surface OXTRs in transfected HEK293T cells, had no effect in hTERT-HM cells. The AVPR1A/2 antagonist YM087, which had no effect on OXTR in HEK293T cells, significantly decreased surface OXTR in hTERT-HM cells (p=0.018). The top two candidate pharmacoperones, SR49059 and L371,257, had similar effects in hTERT-HM cells as in HEK293T cells, increasing cell surface OXTR abundance by 2.3-fold and 2.9-fold, respectively (FIG. 8A). Surface OXTR localization increased by more than twofold after 4 h of treatment with SR49059 or L371,257, reached a plateau after 6 h of treatment, and remained high for 48 h of treatment (FIG. 8B).

To determine the mechanism of action of SR49059 and L371,257, hTERT-HM cells were treated for 12 h with cycloheximide (25 μg/mL), which inhibits protein translation, and brefeldin A (5 μg/mL), which blocks protein transport from the endoplasmic reticulum to the Golgi. Cycloheximide and brefeldin A decreased baseline cell surface OXTR abundance by 18% and 62%, respectively (FIG. 8C; p<0.001, one-sample t test). Concurrent treatment with 10 μM SR49059 had no effect on cell surface OXTR in cells that were also treated with cycloheximide or brefeldin A. L371,257 increased surface OXTR by 20% (p=0.02, one-way ANOVA with Šidák's multiple comparisons) in cells treated with cycloheximide but had no effect on cells treated with brefeldin A (FIG. 8C). Both SR49059 and L371,257 primarily act by mobilizing newly synthesized OXTRs to the cell membrane, and that L371,257 may traffic a small portion of previously synthesized receptors to the cell membrane.

### Example 4: SR49059 and L371,257 Increase Oxytocin-Induced IP1 Production in Immortalized and Primary Human Myometrial Cells

Given the abilities of SR49059 and L371,257 to increase WT OXTR cell surface localization in hTERT-HM cells, it was asked whether these pharmacoperones would also lead to increased oxytocin response in these cells. Oxytocin-induced IP1 production was measured in the absence and presence of these compounds, and it was found that SR49059 and L371,257 increased maximal IP1 production by 37% and 35%, respectively (FIG. 8D).

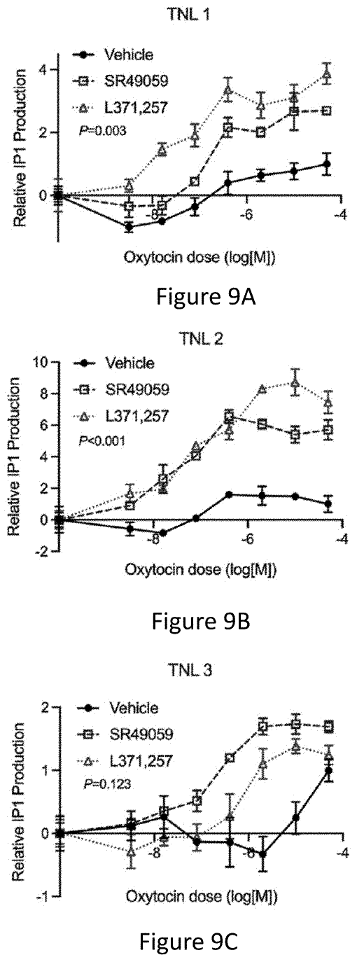

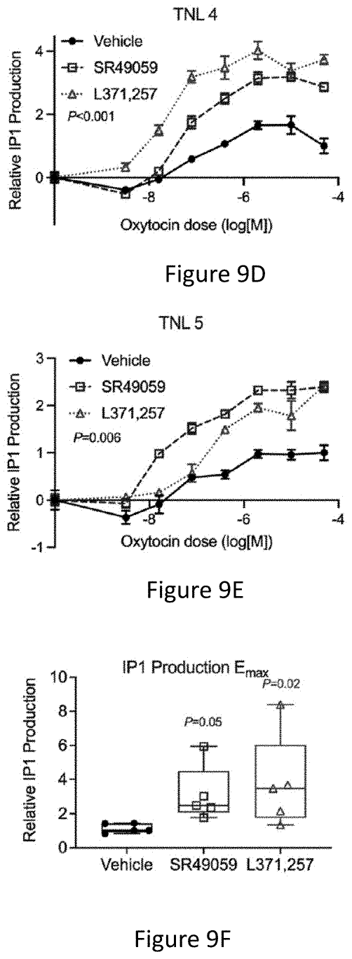

Finally, it was asked whether SR49059 and L371,257 would affect oxytocin response in primary myometrial cells isolated from uterine tissue collected from pregnant patients at the time of term elective Cesarean section. These cells were incubated with SR49059 or L371,257 and quantified IP1 production. In four of the five primary cultures, cells treated with SR49059 and oxytocin accumulated between 1.9 and 7.0 times more IP1 than cells treated with vehicle and oxytocin. Likewise, L371,257 increased response to oxytocin by 1.9- to 5.9-fold (FIG. 9A-9F). Although the magnitude of the effect varied widely, pharmacoperone treatment increased cellular response in primary myometrial cells.

Taken together, the results above indicate that two oxytocin/vasopressin antagonists, SR49059 and L371,257, act as pharmacoperones for both variant and WT OXTR. First, these pharmacoperones restored OXTR trafficking and oxytocin response in HEK293T cells transfected with V281M OXTR, a loss-of-function variant. Second, pharmacoperones mobilized endogenous WT OXTR to the cell surface in a translation-dependent manner. Finally, SR49059 and L371,257 increased response to oxytocin in both hTERT-HM cells and primary human myometrial cells from five individuals.

This data adds to several studies demonstrating the use of pharmacoperones for the closely related AVPR2. Of the AVPR2 pharmacoperones that have been described, the agonists SR49059, YM087, and OPC41061 were included. SR49059 robustly increased cell surface OXTR in HEK293T cells transfected with V281M or WT OXTR, as well as in hTERT-HM cells. However, YM087 and OPC41061 had mixed effects. YM087 did not alter surface OXTR in HEK293T cells but decreased surface OXTR abundance in hTERT-HM cells, whereas OPC41061 increased cell surface OXTR in hTERT-HM cells but not in transfected HEK293T cells. In contrast, the OXTR antagonist L371,257, which has not previously been tested for pharmacoperone activity, had the greatest effect of all the candidate compounds on cell surface OXTR in both transfected HEK293T cells and hTERT-HM cells. It is not known whether L371,257 also acts as a pharmacoperone for AVPR2 or other vasopressin receptors. Given the similarity between members of the oxytocin/vasopressin receptor family, agents that act on OXTR will likely also affect vasopressin receptors, however, the clinical significance of such effects is not known.

Since commercially available OXTR-specific antibodies were not available, examining localization of endogenous, untagged OXTR was not performed. However, SR49059 and L371,257 increased OXTR trafficking to the cell surface regardless of whether OXTR was tagged with an N-terminal HA tag, C-terminal GFP tag, or both. Importantly, the functional effects of SR49059 and L371,257 on oxytocin signaling in primary myometrial cells expressing endogenous, untagged OXTR was assessed.

In the long term, development of OXTR pharmacoperones may improve the safety and effectiveness of oxytocin use during childbirth. Oxytocin is administered to most patients to induce and augment labor and prevent postpartum hemorrhage. However, patients with reduced OXTR function may not benefit from oxytocin treatment. This data show that pharmacoperone treatment can reverse the effects of a loss-of-function OXTR variant, potentially enabling the use of oxytocin in patients with V281M or other variants that disrupt OXTR trafficking.

In addition to their effects on V281M OXTR, SR49059 and L371,257 increased the abundance of WT OXTR on the cell surface in both transfected HEK293T and hTERT-HM cells. This suggests that pharmacoperone treatment can increase oxytocin responsiveness in all patients, regardless of their OXTR genotype. In support of this idea, five primary myometrial samples acquired robust oxytocin responses after treatment with SR49059 and L371,257. Although sequencing data for the patients from whom these samples were obtained was not available, it is unlikely that they harbored the V281M variant, which is most prevalent in the Swedish population and found in 0.2% of non-Finnish Europeans. Instead, they most likely had WT OXTR.

### Example 6: Experimental Procedures

Compounds and Plasmids

Oxytocin stock solutions (Tocris) were diluted to 500 μM in water and stored at −80° C. until just before use. Stock solutions for all candidate pharmacoperones (Table 1, Tocris) were diluted to 5 mM in DMSO and stored at −80° C. until just before use. Cycloheximide (Millipore Sigma) was diluted to 10 mg/ml in DMSO and stored at −20° C. until just before use. Brefeldin A stock solution (5 mg/ml) was obtained from Biolegend. An equivalent volume of DMSO was used as a vehicle control for all experiments. Plasmids encoding WT and V281M OXTR, OXTR-GFP, and HA-OXTR-GFP were generated as previously described (Malik, et al.; ACS Pharmacol. Transl. Sci. 2021; 4:1543-1555).

Cell Lines

All cell lines were maintained in Dulbecco's Modified Eagle Medium (DMEM)/Ham's F12 media without phenol red supplemented with 10% fetal bovine serum (FBS) and 25 μg/ml gentamicin. Cells were kept in a humidified cell culture incubator at 37° C. with 5% CO₂. Stably transfected HEK293 cell lines expressing WT and V281M OXTR-GFP were selected and maintained with 500 μg/ml G418 (Millipore Sigma). The hTERT-HM cell line was from Dr Jennifer Condon at Wayne State University.

Primary Human Myometrial Cell Cultures

Human myometrial tissue samples from the lower uterine segment were obtained from nonlaboring patients at >37 weeks' gestation during Cesarean section under spinal anesthesia. Human subjects research was performed in accordance with the principles stated in the Declaration of Helsinki. Participants signed consent forms approved by the Washington University in St Louis Internal Review Board (protocol no. 201108143). Tissues were cut into small pieces and incubated with 1 mg/ml collagenase IA and collagenase XI (Millipore Sigma) for 45 to 60 m at 37° C. with rotation. The collagenase-treated mixture was then passed through a 70 μm cell strainer, and collagenase was neutralized with 10% FBS. Cells were centrifuged, resuspended, and plated in DMEM/Ham's F12 media without phenol red, supplemented with 5% Smooth Muscle Cell Growth Medium 2 (PromoCell). Cells were used at passage 0, 1, or 2.

Quantitative Flow Cytometry

HEK293T cells were plated in T25 flasks (106 cells/flask) and transfected the next day with 300 ng of plasmid DNA encoding HA-OXTR-GFP and 4 μl of TransIT-LT1 reagent (Mirus). Eight hours after transfection, candidate pharmacoperone or vehicle was added to each flask to a final concentration of 10 μM. Sixteen hours later, cells were detached with CellStripper (Corning) and analyzed by flow cytometry.

The Genome Engineering and iPSC Center at Washington University in St Louis used CRISPR-Cas9 to introduce an HA tag at the N-terminus of the OXTR gene in hTERT-HM cells. After drug incubation, cells were detached with TrypLE Express (Thermo Fisher) before flow cytometry.

Labeling and flow cytometry quantitation of cell surface HA-OXTR-GFP were performed as previously described (Malik, et al.; ACS Pharmacol. Transl. Sci. 2021; 4:1543-1555; Chen, et al.; Methods Mol. Biol. 2017; 1570:117-138). An empirically determined saturating concentration of phycoerythrin-conjugated anti-HA antibody (901518, Biolegend) was used for each cell type (16 ng/μl for transfected HEK293T cells, 10 ng/μl for hTERT-HM cells).

Immunofluorescence

HEK293T cells stably transfected with V281M OXTR-GFP were plated in poly-D-lysine-coated 8-well culture slides. After drug treatment, cells were washed once with PBS, then fixed with 4% paraformaldehyde for 20 min at room temperature. Cells were washed once with PBS, incubated in blocking and permeabilization buffer (5% FBS in PBS plus 0.1% Tween-20) for 1 h at room temperature, then incubated with anti-PDI and anti-golgin-97 antibodies (C81H6 and D82PK, respectively, 1:100, Cell Signaling Technologies) overnight at 4° C. Cells were washed three times with PBS plus 0.1% Tween-20, then incubated with goat anti-rabbit secondary antibody (1:1000, AlexaFluor 555, Thermo Fisher) for 1 h at room temperature. Cells were washed and nuclei labeled with NucBlue fixed cell stain (Thermo Fisher) before imaging by confocal microscopy (Leica DM4000). Investigators were masked to treatment when imaging slides. Colocalization (Pearson's r) was calculated by using the JaCoP plugin in ImageJ software.

IP1 Production

IP-One Gq Homogeneous Time Resolved Fluorescence kit (Cisbio) was used according to the manufacturer's instructions to measure IP1 production. To measure IP1 production in transfected HEK293T cells, cells were transfected in T25 flasks as above. Eight hours later, cells were treated with 10 μM SR49059, 10 μM L371,257, or 0.2% DMSO (vehicle). Sixteen hours later, cells were washed extensively and detached with TrypLE Express. Cell suspensions were incubated with the indicated doses of oxytocin (60,000 cells in a total volume of 15 μl cell suspension per well) for 1 h at 37° C. After lysis and addition of Homogeneous Time Resolved Fluorescence donor and acceptor, plates were incubated for 1 h at room temperature. Fluorescence was read on a PerkinElmer Envision plate reader. The same protocol was used to measure IP1 production in hTERT-HM cells and primary human myometrial cells, but 5000 to 7500 cells were used per well.

Nonlinear regression with least-squares fitting was used to generate dose-response curves with the following model:

Y=Bottom+(Top−Bottom)/(1+10\hat{ }((Log EC50 or IC50−X)*HillSlope))(GraphPadPrism)

In this model, Y=response, X=log(oxytocin concentration), and no constraints were placed on any values. Emax values were compared by performing nested extra sum-of-squares F-tests as previously described (Hall, et al.; Br. J. Pharmacol. 2010; 161:1276-1290; Meddings, et al.; Am. J. Physiol. 1989; 257:G982-989).

1. A method of increasing the display of oxytocin receptor (OXTR) on a plasma membrane in a cell of a subject comprising: administering an OXTR chaperone, wherein the OXTR chaperone increases OXTR on a cell surface.

2. A method of increasing or restoring oxytocin sensitivity in a subject comprising: administering an OXTR chaperone, wherein the OXTR chaperone increases OXTR on a cell surface.

3. A method of increasing the efficacy of oxytocin or synthetic oxytocin in a subject comprising: administering an OXTR chaperone, wherein the OXTR chaperone increases OXTR on a cell surface.

4. The method of claim 1, further comprising administering oxytocin or a derivative thereof, such as synthetic oxytocin (e.g., Pitocin) to the subject.

5. The method of claim 1, wherein the OXTR chaperone is selected from an OXTR binding agent (e.g., OXTR antagonist) or vasopressin inhibitor.

6. The method of claim 1, wherein the OXTR chaperone is an OXTR antagonist, L371,257 (1-[1-[4-(1-acetylpiperidin-4-yl)oxy-2-methoxybenzoyl]piperidin-4-yl]-4H-3,1-benzoxazin-2-one).

7. The method of claim 1, wherein the OXTR chaperone is a vasopressin inhibitor, SR49059 ((2S)-1-[[(2R,3S)-5-Chloro-3-(2-chlorophenyl)-1-[(3,4-dimethoxyphenyl)sulfonyl]-2,3-dihydro-3-hydroxy-1H-indol-2-yl]carbonyl]-2-pyrrolidinecarboxamide).

8. The method of claim 1, wherein the OXTR chaperone is a vasopressin inhibitor, OPC 21268 (N-[3-[4-[[4-(3,4-dihydro-2-oxo-1(2H)-quinolinyl)-1-piperidinyl]carbonyl]phenoxy]propyl]-acetamide).

9. The method of claim 1, wherein the OXTR chaperone is a vasopressin inhibitor, SSR 149415 ((2S,4R)-1-[(3R)-5-Chloro-1-[(2,4-dimethoxyphenyl)sulfonyl]-2,3-dihydro-3-(2-methoxyphenyl)-2-oxo-1H-indol-3-yl]-4-hydroxy-N,N-dimethyl-2-pyrrolidinecarboxamide).

10. The method of claim 1, wherein the OXTR chaperone is a vasopressin inhibitor, tolvaptan (N-(4-{[(5R)-7-Chloro-5-hydroxy-2,3,4,5-tetrahydro-1H-1-benzazepin-1-yl]carbonyl}-3-methylphenyl)-2-methylbenzamide).

11. The method of claim 1, wherein increasing the display of OXTRs comprise increasing the trafficking of the receptors from intracellular stores (in the endoplasmic reticulum and Golgi body) to the cell surface.

12. The method of claim 1, wherein the OXTR chaperone is administered in an amount effective to:
improve trafficking of the oxytocin receptor;
enhance clinical response to oxytocin;
increase uterine contractions during childbirth;
induce or augment labor;
prevent or reduce risk of postpartum hemorrhage;
sensitize cells to the oxytocin; or
increase OXTR signaling.

13. The method of claim 12, wherein the OXTR chaperone is administered in an amount effective to reduce risk of adverse events, such as cesarean section, uterine atony, or post-partum hemorrhage.

14. The method of claim 1, wherein the OXTR chaperone is administered in an amount effective to modulate social behavior.

15. The method of claim 1, wherein the OXTR chaperone is administered in an amount effective to modulate lactation.

16. The method of claim 1, wherein the subject has oxytocin insensitivity.

17. The method of claim 1, wherein the subject has loss-of-function (variants that would normally impair OXTR trafficking and decrease oxytocin response) OXTR genetic variants (e.g., V281M, E339K).

18. The method of claim 1, wherein the subject has or is suspected of having autism spectrum disorder.

19. The method of claim 1, wherein the subject has or is suspected of having a psychiatric condition.

20. The method of claim 1, wherein the subject has pain or is in need of pain relief.
